(19) United States
(12) Patent Application Publication
(10) Pub. No.: US 2024/0382371 A1
(43) Pub. Date: Nov. 21, 2024

(54) SYSTEM AND METHOD FOR PREVENTION OF DEEP-VEIN THROMBOSIS (DVT) EDEMA OR MUSCLE ATROPHY

(71) Applicant: Restorative Therapies, Inc., Nottingham, MD (US)

(72) Inventors: Scott Simcox, Baltimore, MD (US); Wendy Warfield, Seven Valleys, PA (US); James Janicki, Pheonix, MD (US)

(21) Appl. No.: 18/667,893

(22) Filed: May 17, 2024

Related U.S. Application Data
(60) Provisional application No. 63/637,166, filed on Apr. 22, 2024.
(60) Provisional application No. 63/467,701, filed on May 19, 2023.

(51) Int. Cl. A61H 9/00; A61N 1/04; A61N 1/36

(52) U.S. Cl. CPC A61H 9/0092; A61N 1/0452; A61N 1/3603; A61H 2201/0103; A61H 2201/10; A61H 2201/1238; A61H 2201/1409; A61H 2201/1642; A61H 2201/165; A61H 2201/5007; A61H 2201/5064

(57) ABSTRACT

In a first aspect, a system for prevention of muscle atrophy is presented. The system includes an attachable device. The attachable device includes at least one electrode configured to stimulate a portion of a leg of a subject. The attachable device includes an air bladder configured to apply pressure to the portion of a leg of a subject. The attachable device includes an air inlet port fluidically connected to the air bladder. The controller includes a compression pump. The stimulation unit includes at least one air tube connecting an outlet of the compression pump to the air inlet port of the attachable device. The controller includes a stimulation unit. The stimulation unit is configured to stimulate at least a portion of a leg of a subject.

### CROSS-REFERENCE TO RELATED APPLICATIONS

This application claims priority to, and the benefit of U.S. Provisional App. No. 63/637,166, filed Apr. 22, 2024, and U.S. Provisional App. No. 63/467,701, filed May 19, 2023, both applications incorporated by reference herein in their entirety.

### TECHNICAL FIELD

This disclosure relates to muscle atrophy that may be acquired through prolonged periods of bed rest. In particular, this disclosure relates to systems and methods of prevention of muscle atrophy.

### BACKGROUND

Prolonged sedentary bed rest creates physical, cognitive, and mental health impairments. Additionally, prolonged sedentary bed rest may cause intensive care unit (ICU) acquired weakness. ICU acquired weakness is a skeletal muscle disorder that commonly occurs in critically ill patients. ICU acquired weakness is one of the most important and common result of critical illness, which can affect up to 70% of ICU survivors and is often long-lasting.

### SUMMARY

In a first aspect, a system for prevention of amelioration of deep-vein thrombosis (DVT) edema or muscle atrophy is presented. The system includes an attachable device. The attachable device includes at least one electrode configured to stimulate a body part of a subject. The attachable device includes an air bladder configured to apply pressure to the portion of a leg of a subject. The attachable device includes an air inlet port fluidically connected to the air bladder. The system includes a compression pump and a controller. The controller includes at least one air tube connecting an outlet of the compression pump to the air inlet port of the attachable device. The controller includes a stimulation unit. The stimulation unit is configured to stimulate a body part of a subject.

In another aspect, a method of preventing amelioration of deep-vein thrombosis (DVT) edema or muscle atrophy is presented. The method includes placing an attachable device on body part of a subject. The attachable device includes at least one electrode configured to stimulate a body part of a subject. The attachable device includes an air bladder configured to apply pressure to a body part of a subject. The attachable device includes an air inlet port fluidically connected to the air bladder. The method includes selecting an operation mode of a controller of a stimulation unit in communication with the attachable device. The method includes applying stimulation to a body part of a subject through at least one electrode, applying pressure to the body part through an air bladder, or both, based on an operation mode. through the stimulation unit based on the operation mode.

### BRIEF DESCRIPTION OF THE DRAWINGS

These and other features, aspects, and advantages of the present disclosure will become better understood with regard to the following description and accompanying drawings.

FIG. 1 illustrates a block diagram of an exemplary embodiment of a system for prevention of deep-vein thrombosis (DVT) edema or muscle atrophy;

FIG. 2 is an illustration of a system for prevention of deep-vein thrombosis (DVT) edema or muscle atrophy;

FIG. 3 is an illustration of an attachable device;

FIG. 4 illustrates an embodiment of a resistance device that may be used with the present system;

FIG. 5A illustrates an embodiment of a flexed leg of a patient using the present system;

FIG. 5B illustrates an embodiment of an extended leg of a patient using the present system;

FIG. 6 illustrates a top view of a system for prevention of muscle atrophy;

FIG. 7 illustrates a flowchart of a method of preventing deep-vein thrombosis (DVT) edema or muscle atrophy; and

FIG. 8 is a diagram of a computing device that may be implemented in systems and methods described throughout this disclosure.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

Aspects of the present disclosure can be used to prevent or reduce the severity of deep-vein thrombosis (DVT) edema of an individual and/or ICU acquired muscle weakness. In some embodiments, aspects of the present disclosure may include an attachable device or other type of wearable with a plurality of stimulators and one or more air bladders disposed within the attachable device. An attachable device may be attached to a leg of a patient, for example. More generally, the attachable device may be positioned to stimulate muscles that cause movement about a joint. A stimulation unit in communication with the attachable device may operate the electrodes and air bladders which may help prevent DVT edema. In some embodiments, one or more resistance devices may be employed. Employing one or more resistance devices may include connecting a controller to the one or more resistance devices and providing resistance to a subject's body part through the resistance devices. One or more resistance devices may be in communication with a controller and/or stimulation unit of a controller and may provide resistance to a body part of a subject, such as, but not limited to, a limb, finger, ankle, wrist, hip, neck, toe, and/or any other body parts that the attachable device is positioned to stimulate. A controller of a stimulation unit may operate the one or more resistance devices, electrodes, and/or air bladders. In some embodiments, one or more resistance devices in communication with a stimulation unit may be configured to provide a force opposing a joint movement of a subject that may be caused by one or more stimulators delivering current to muscles of the subject that bring about movement about the joint. Various operation modes of a controller of a stimulation unit may also be used to prevent or ameliorate DVT edema or treat the other conditions or cause the stimulations of muscles or body part movements as provided for herein.

As used herein, the terms “subject” and “patients” can be used interchangeably. In some embodiments, the systems and methods are utilized with, or performed on, subject's that are sedated or unconscious. In some embodiments, the subject is in a coma, which can be, for example, a medically induced coma.

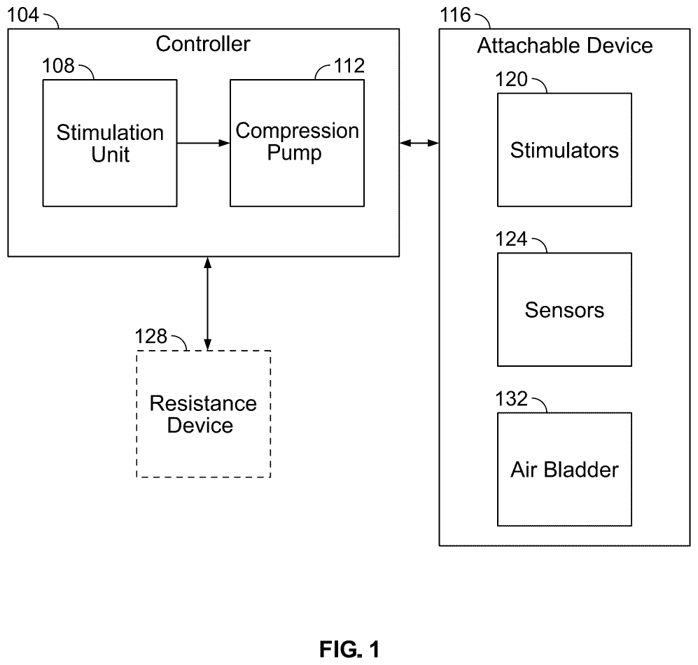

Referring now to FIG. 1, system 100 for prevention of deep-vein thrombosis (DVT) edema or muscle atrophy is presented. “Muscle atrophy” refers to the deterioration of muscles due to lack of use. Muscle atrophy can occur in patients that are bed ridden for prolonged periods of time. Muscle atrophy may include ICU acquired weakness. ICU acquired weakness is a skeletal muscle disorder that commonly occurs in critically ill patients. ICU acquired weakness can cause deep vein thrombosis (DVT) edema. System 100 may be used to prevent and/or reduce the severity of DVT edema.

System 100 may include controller 104. Controller 104 may include one or more processors, memories, and the like. Controller 104 may include stimulation unit 108. Stimulation unit 108 may be any processing device, such as, but not limited to, microcontroller, microprocessor, system on a chip (SoC), and/or other computing devices. Stimulation unit 108 may be configured to communicate with compression pump 112, stimulators 120, sensors 124, and/or resistance device 128, such as through a wired or wireless connection.

In some embodiments, system 100 may include attachable device 116. An “attachable device” as used in this disclosure is a device that can be secured to a subject's body part. For instance, attachable device 116 may attach to a leg, arm, hand, or other body part of a subject. Attachable device 116 may be attachable to a body part of a subject through Velcro, straps, hooks, and/or other attachment devices. In some embodiments, attachable device 116 may wrap around or otherwise attach to a subject's leg, arm, hand, or other body part. Attachable device 116 may be a cuff, armband, sleeve, or other wearable device. Attachable device 116 may include stimulators 120. A “Stimulator” as used in this disclosure is any device capable of delivering a current to a body part. Stimulators 120 may include one or more electrodes. In some embodiments, stimulators 120 may be 6-channel stimulators or other types of stimulators. In some embodiments, one or more electrodes of stimulators 120 may include an electrode pad. An electrode pad may be adhesive and may deliver a current to an area of a subject's body. In some embodiments, attachable device 116 may have stimulators 120 placed at various areas within and/or around attachable device 116. Attachable device 116 may have one or more electrical connectors that may provide electrical communication from an external power source to one or more stimulators 120. For instance, attachable device 116 may have an electrical connection port that be in electrical communication with one or more stimulators 120. An electrical connection port may be connectable to one or more power cables, which may be connected to stimulator unit 104. In some embodiments, attachable device 116 may include about 12 stimulators 120, greater than 12 stimulators 120, less than 12 stimulators 120, and/or other quantities of stimulators 120. Stimulators 120 may be positioned around various portions of attachable device 116. For instance, six stimulators 120 may be positioned at a top of attachable device 116 and six stimulators 120 may be positioned at a bottom of attachable device 116. In some embodiments, attachable device 116 may include sets of stimulators 120, such as sets of two, three, and/or other quantities of stimulators 120.

In some embodiments, attachable device 116 may include one or more air bladders 132. Air bladder 132 may be made of an expandable and/or stretchable material, such as rubber, plastic, and/or other materials. Air bladder 132 may be positioned within and/or at a side of attachable device 116. Air bladder 132 may be positioned over or under one or more stimulators 120. In some embodiments, attachable device 116 may include a first air bladder 132 positioned at a top portion of attachable device 116 and a second air bladder 132 positioned at a bottom portion of attachable device 116.

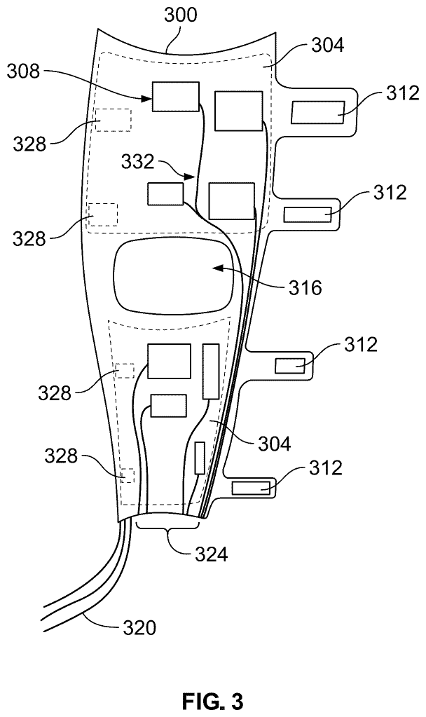

Attachable device 116 may include one or more sensors 124. A “sensor” as used in this disclosure is any device capable of detecting physical changes in an object and/or environment. Physical changes may include, but are not limited to, temperatures, pressures, currents, voltages, and the like. For instance, sensor 124 may include current sensors, voltage sensors, hall effect sensors, tactile sensors, temperature sensors, and/or other types of sensors. Attachable device 116 may include a plurality of sensors 124. A plurality of sensors 124 may include a same sensor type or may be a combination of two or more various sensing devices, such as, but not limited to, a pressure sensor and a current sensor. Attachable device 116 may be explained in greater detail below with reference to FIG. 3.

Referring still to FIG. 1, controller 104 may include compression pump 112. Compression pump 112 may be configured to compress air at various pressures and/or volumes. For instance, and without limitation, compression pump 112 may include a continuous compression, sequential compression, gradient compression, intermittent compression, cryocompression, or other compression pump device. In some embodiments, compression pump 112 may be a pneumatic or other compression pump. Compression pump 112 may be configured to input ambient air and output compressed air, such as at about 10 pounds per square inch (PSI), greater than 10 PSI, or less than 10 PSI. Compression pump 112 may be in fluidic communication with air bladder 132 of attachable device 116. A “fluidic communication” as used in this disclosure is a form of connection between two or more fluidic bodies in which mass of one fluidic body is received at a second fluidic body, and vice versa. For instance and without limitation, compression pump 112 may be in fluidic communication with one or more air bladders 132 through one or more air tubes. An air tube may be a tubing that connects an air outlet of compression pump 112 to an air inlet of air bladder 132. An “air outlet” as used in this disclosure is an exit port of a device in which fluid leaves. An “air inlet” as used in this discourse is an entry port of a device in which fluid enters. Compression pump 112 may provide compressed air at varying PSIs to one or more air bladders 132 through one or more air tubes or other fluidic connection devices. As a non-limiting example, a first air tube may connect a first air outlet of compression pump 112 to an air inlet of a first air bladder 132, and a second air tube may connect a second air outlet of compression pump 112 to an air inlet of a second air bladder 132.

In some embodiments, attachable device 116 may include two or more air bladders 132, which may be positioned along key areas of attachable device 116. Key areas may include, but are not limited to, parts of a leg, parts of an arm, and the like. For instance and without limitation, a key area placement of air bladder 132 on attachable device 116 may be at a lower portion of attachable device 116 designed to interact with a side of a calf muscle of a subject. Attachable device 116 may have a first air bladder 132 positioned at a top portion of attachable device 116, such as above a knee hole of attachable device 116, and a second air bladder 132 positioned at a bottom portion of attachable device 116, such as below a knee hole of attachable device 116. A knee hole of attachable device 116 may be an opening of attachable device 116 which may allow movement of a subject's knee, such as extension of a subject's knee. In some embodiments, first air bladder 132 may include two or more air bladders.

Stimulation unit 108 of controller 104 may be configured to command one or more stimulators 120 to deliver a current, voltage, waveform, or the like to a subject. Stimulation unit 108 may command one or more stimulators 120 based on sensor data received from sensors 124. Sensor data may include, but is not limited to, voltages, currents, accelerations, and the like. Stimulators 120, in some exemplary embodiments, may deliver a current to induce stimulation od muscles anywhere between about 0 milliamps to about 140 milliamps, a pulse width of about 50 us to about 3,000 μs, and/or a frequency of about 10 Hz to about 100 HZ. A waveform delivered by stimulators 120 may be alternating, monophasic, or other types of waveforms. “Monophasic” refers to a type of waveform that does not cross the zero-flow baseline. Stimulation unit 108 of controller 104 may be configured to command compression pump 112 to apply a pressure to a subject's body part via air bladder 132 of attachable device 116, in some embodiments. For instance compression pump 112 may apply pressure to a subject's body part via air bladder 132 of attachable device 116 which may increase a blood flow to the body part. Stimulation unit 108 may adjust pressures delivered to air bladder 132 via compression pump 112 based on sensor data received from sensors 124. In some embodiments, a pressure detected by sensors 124 may be too low, to which controller 104 may command compression pump 112 to increase a pressure to a subject's body part via air bladder 132. In other embodiments, a pressure detected by sensors 124 may be too high, to which controller 104 may command compression pump 112 to decrease a pressure to a subject's body part via air bladder 132. High and low pressures may be determined by a medical professional and received via subject input and/or external computing devices in communication with controller 104. In some embodiments, controller 104 may automatically adjust pressures applied to a body part of a subject based on sensor data generated by sensors 124. For instance and without limitation, sensor data may include pressures of one or more air bladders 132. Stimulation unit 108 may adjust pressures of air bladder 132 based on sensed pressures of air bladder 132. In some embodiments, stimulation unit 108 may be configured to adjust and/or operate stimulators 120 and compression pump 112 in combination, which may deliver a combination of pressures via air bladder 132 and electrical stimulation via stimulators 120 to a subject through attachable device 116. For instance and without limitation, stimulation unit 108 may intermittently increase electrical stimulation via stimulators 120, pressures via air bladders 132, and the like. Intermittently increasing electrical stimulation and/or pressures may increase blood flow and/or muscle stimulation of a subject's body part. In some embodiments, stimulation unit 108 may increase a pressure of air bladder 132 while decreasing a current of stimulators 120. In other embodiments, stimulation unit 108 may increase a current of stimulators 120 while decreasing a pressure of air bladder 132. In some embodiments, stimulation unit 108 may increase both a current of stimulators 120 and a pressure of air bladder 132. In some embodiments, stimulation unit 108 may decrease a current of stimulators 120 and a pressure of air bladder 132. Stimulation unit 108 may be configured to individually operate one or more stimulators 120, air bladders 132, and the like. As a non-limiting example, in 3 sets of two stimulators 120, stimulation unit 108 may increase a current of two stimulators 120 while keeping a current unchanged of the rest of the stimulators 120. Likewise, and continuing this non-limiting example, stimulation unit 108 may be configured to increase a pressure of a first air bladder 132 while decreasing a pressure of a second air bladder 132. One of ordinary skill in the art, upon reading this disclosure, will appreciate the many various combinations of pressures and electrical stimulations that may be applied via stimulation unit 108.

Referring still to FIG. 1, in some embodiments, controller 104 and/or stimulation unit 108 may be in communication with resistance device 128. A “resistance device” as used in this disclosure is any device that provides force in opposition to a subject's movements. Resistance device 128 may include, but is not limited to, a foot pedal, cycling machine, and/or other device. Resistance device 128 may be configured to provide an opposing force to a limb of a subject attachable device 116 may be wrapped around. As a non-limiting example, resistance device 128 may be configured to provide a force against a subject's extension of an ankle, quadriceps, hip muscles, chest muscles, back muscles, neck muscles, arm muscles, and/or other body part.

In some embodiments, stimulation unit 108 may be configured to operate in one or more modes of operation. Modes of operation may include, but are not limited to, automated intermittent pneumatic compression (IPC), exercise, electrical stimulation, and/or other modes. In an IPC mode, stimulation unit 108 may be configured to adjust a pressure of one or more air bladders 132, such as in intermittent periods. In an exercise mode, stimulation unit 108 may be configured to activate a subject's leg muscles through electrical stimulation provided by stimulators 120 while opposing activation of the subject's leg muscles through a force provided by resistance device 128. In some embodiments, in an exercise mode, compression pump 112 may be deactivated or otherwise shut off. For instance, and without limitation, a subject may only want to exercise a body part without utilization of compression delivered via compression pump 112. In some embodiments, one or more parameters of an exercise mode may be adjustable. For instance, a nurse or other healthcare provider may select an exercise mode that includes plantar and/or dorsi flexion of an ankle ji, flexion and/or extension of a knee joint, and/or a combination of both. In some embodiments, one or more parameters of an exercise mode that may be selectable may include repetitions, resistance levels, rest time, sets, durations, and the like. In some embodiments, an exercise mode of stimulation unit 108 may include an exercise regime, which may be set by one or more healthcare providers. For instance, an exercise regime may include exercising both legs of a subject in an alternating pattern. An electrical stimulation mode of operation may include activating one or more leg muscles of a subject. For instance, an electrical stimulation mode of operation may include activating quadriceps, hamstrings, plantar, dorsi, and/or other muscles of a subject. In some embodiments, stimulation unit 108 may selectively activate one or more stimulators 120 which may selectively activate one or more muscles of a leg of a subject. An electrical stimulation mode may include one or more parameters, such as, but not limited to, duration, intensity, voltages, amps, waveforms, and the like. An electrical stimulation mode of operation may be combined with an exercise mode of operation, in some embodiments.

In some embodiments, controller 104 may apply electrical stimulation through stimulators 120, which may cause a subject's muscles to contract, and simultaneously activate resistance device 128 to counteract the subject's muscle contraction. By activating stimulators 120 and resistance device 128, controller 104 and/or stimulation unit 108 may be able to exercise a subject's body part without their active control. Controller 104 may determine a force generated by a subject's body part through involuntary contraction of the subject's body part attributed to stimulators 120 and may calculate an opposing force to be applied through resistance device 128. As a non-limiting example, a subject may have their quadricep contracted by electrical stimulation applied from stimulators 120, which controller 104 may determine, through sensors 124, to be about 10 lbs of force. Controller 104 may calculate an opposing force of about 5 lbs to about 8 lbs and apply this range of opposing force to the subject's body part through resistance device 128. Controller 104 may adjust opposing forces generated by resistance device 128 over time based on a subject's increased muscle strength gained due to resistance device 128 opposing contraction of a subject's body part due to electrical stimulation by stimulators 120.

In some embodiments, parameters of electrical stimulation of stimulators 120 and opposing forces of resistance device 128 may be set by a medical professional, controller of the system, and/or the subject that the system is being utilized with. In some embodiments the parameters are set by a medical professional. In some embodiments, the parameters are not set by the subject. In other embodiments, controller 104 may automatically calculate an opposing force to be generated by resistance device 128 based on patient data, sensor data generated by sensors 124, and/or other data. For instance, controller 104 may determine that a subject may need an increase in resistance and may increase a resistance provided by resistance device 128 while applying a same electrical stimulation to the subject through stimulators 120. Likewise, controller 104 may determine that a subject may need a decrease in resistance and may decrease a resistance provided by resistance device 128. Controller 104 may generate exercise regimes, which may include repetitions, sets, resistances, and/or other parameters. Controller 104 may automatically calculate sets, repetition, resistances, and/or other parameters of an exercise regime based on patient data, data generated by sensors 124, and/or other data. Controller 104 may increase a resistance between sets, decrease a resistance between sets, increase a repetition range, decrease a repetition range, increase a duration of a repetition, decrease a duration of a repetition, increase a rest time between reps and/or sets, decrease a rest time between reps and/or sets, and/or modify other parameters of an exercise regime. Exercise regimes may be muscle specific, such as specific to quadricep muscles, bicep muscles, triceps muscles, pectoral muscles, back muscles, ankle muscles foot muscles, and/or other muscles. In some embodiments, controller 104 may provide a cardio exercise to a subject through resistance devices 128 and/or stimulators 120. For instance, and without limitations, controller 104 may apply short duration repetitions over a period of time which may cause a higher frequency of resisted contraction of a subject's muscles, providing a form of aerobic exercise. Aerobic exercise may help increase a subject's cardiovascular health.

Stimulation unit 108 may change a waveform, current, voltage, and the like based on received sensor data from sensors 124. In some embodiments, stimulation unit 108 may be configured to interpret sensor data to determine muscle activity, blood flow, and the like of a body part attachable device 116 may be attached to. Based on sensor data interpreted from data received from sensors 124, stimulation unit 108 may automatically adjust one or more parameters of stimulation applied, through stimulators 120, air bladder 132, and/or resistance device 128. Controller 104 and/or stimulation unit 108 may operate in a closed-loop system. A “closed-loop system” refers to an automatic control system in which an operation, process, or mechanism is regulated by feedback. Stimulation unit 108 may change one or more parameters of stimulators 120, air bladder 132, and/or resistance device 128 based on sensor data generated by sensors 124. As a non-limiting example, sensor data generated by sensors 124 may indicate inadequate blood flow to a portion of a subject's leg, to which controller 104 may increase a pulse, pressure, or other parameter of air bladder 132. Likewise, and continuing this example, sensor data generated by sensors 124 may indicate that there is low muscle responsiveness in a subject's leg, to which stimulation unit 108 may increase a voltage, current, frequency, or other parameter delivered by stimulators 120. Parameters of a closed-loop system of controller 104 and/or stimulation unit 108 may be received via medical professional's input and may be subject specific, therapy specific, recovery program specific, and the like.

For instance, based on a patient's diagnosis, time in bed, height, weight, age, and/or other parameters, controller 104 may adjust electrical stimulation, applied pressure, and/or exercise modes of resistance devices 128. Sensors 124 may generate data that controller 104 may generate a trend over time from, which may be a trend of exercise per day, a trend of muscle strength increase, and/or other trends. Controller 104 may adapt electrical stimulation, pressures, and/or exercise provided by resistance devices 128 based on one or more trends of a patient generated by controller 104. In some embodiments, a closed-loop system may be based on parameters for a specific therapy and/or recovery program for a subject. For instance, electrical stimulation, pressures applied, and/or exercise provided by resistance device 128 may have one or more values set by a medical professional for a specific therapy and/or recovery regime for a patient. A therapy and/or recovery program or regime for a patient may include a specific order of electrical stimulation, pressures applied, and/or exercise provided by resistance devices 128. For instance, electrical stimulation may be applied first, followed by pressure from air bladder 132, followed by exercise provided by resistance device 128. Any order of electrical stimulation, pressures, and exercise provided by resistance device 128 may be implemented, without limitation. A therapy and/or recovery program or regime may have various settings of stimulations, such as duration of electrical stimulation, intensity of electrical stimulation, frequency of electrical stimulation, duration of pressures applied, intensity of pressures applied, frequency of pressures applied, duration of exercises, intensity and/or resistance of exercises, and/or frequency of exercises. In some embodiments, controller 104 and/or stimulation unit 108 may automatically adjust and/or determine parameters and/or weights associated with parameters of a closed-loop system. Controller 104 may be configured to automatically generate a therapy and/or recovery program for a patient based on patient data and/or diagnosis. Patient data may include, but is not limited to, age, weight, sex, and/or other demographic data. Diagnosis may include any medical condition that may require long periods of bed rest, such as, but not limited to, fractures, broken bones, infections, lung conditions, heart conditions, brain conditions, and/or other medical issues. Controller 104 may set one or more parameters of electrical stimulation, pressures applied, and/or exercise based on patient data and/or medical conditions. Controller 104 may be configured to train and/or run a machine learning model. A machine learning model may be trained with training data correlating sensor data to one or more parameters of stimulation. Training data may be received via user input, external computing devices, and/or previous iterations of processing. A machine learning model may be trained to input patient data which may include a diagnosis and output a therapy and/or recovery program. Controller 116 may utilize any type of machine learning model, without limitation, such as, but not limited to, logistic regression, decision tree, k-nearest neighbors, random forest, k-means clustering, and/or other types of models.

Various information may be displayed through a display device in communication with Controller 104. Controller 104 may be in communication with a display device via a wired or wireless connection. A display device may include, but are not limited to, monitors, smartphones, laptops, tablets, and the like. In some embodiments, controller 104 may be configured to receive a person's (e.g., medical professional, system controller, robot, or subject) input through one or more user interfaces (UI) of one or more display devices. For instance and without limitation, a person (e.g., medical professional, system controller, robot, or subject) may select one or more modes of operation of stimulation unit 108 through a UI of a display device. In some embodiments, controller 104 may display information such as, but not limited to, pressures of compression pump 112 and/or air bladders 132, currents and/or voltage of stimulators 120, an indication of which stimulators 120 and/or air bladders 132 are currently activated, force applied by resistive device 128, trends in muscle strength of a subject, and/or other information. A person (e.g., medical professional, system controller, robot, or subject) may select one or more parameters of electrical stimulation, pressure, and/or exercise provided by resistance device 128. In some embodiments, based on data collected from previous stimulation, controller 104 may display recommended stimulations for a subject, such as electrical, pressures, and/or exercises. A person (e.g., medical professional, system controller, robot, or subject) may select or deny a recommended stimulation, which may be used as input for controller 104 to provide better recommendations over time.

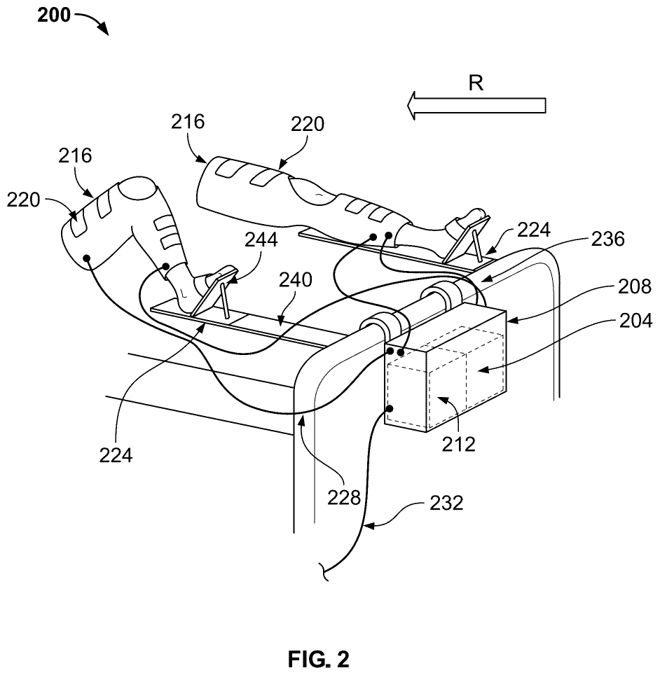

Referring now to FIG. 2, a perspective view of a system 200 for prevention of deep-vein thrombosis (DVT) edema or muscle atrophy is presented. System 200 may include attachable devices 216, stimulators 220, air bladders (not shown in FIG. 2), stimulator unit 204, compression pump 212, stimulation unit 208, and/or resistance device 224 as described above with reference to FIG. 1.

As shown in FIG. 2, two attachable devices 216 may be used, one attachable device 216 for each leg of a subject. In some embodiments, system 200 may interact with one leg of a subject. In other embodiments, system 200 may interact with both legs of a subject. Controller 204 may be powered by power cable 232. Power cable 232 may provide power to stimulation 204 from one or more external power sources, such as, but no limited to, batteries, wall outlets, and the like. Air tubes 228 may connect compression pump 212 to one or more air bladders of attachable devices 216. Stimulation cables 236 may provide currents and/or voltages from controller 204 and/or stimulation unit 208 to one or more stimulators 220 of attachable devices 216. Stimulation unit 208 may be configured to operate simulators 220, air bladders of attachable devices 216, and/or other devices of system 200, similar to system 100 as described above with reference to FIG. 1.

In some embodiments, system 200 may include resistance device(s) 224. In an embodiment as shown in FIG. 2, resistance devices 224 may be a foot pedal moveable along a track. Resistance devices 224 may provide a force in direction R, such as through one or more foot pedals, without limitation. A force in direction R may oppose a force of a leg of a subject, which may provide resistance to one or more muscles of the leg of the subject. Controller 204 may include one or more motors that may be configured to operate resistance devices 224. Resistance devices 224 may include a track 240 with a foot pedal 244 slidable along the track 240. A foot pedal of resistance devices 224 may be positioned at one or more angles relative to a leg of a subject in a resting position, such as, but not limited to anywhere between about a 0 degree angle to about a 180 degree angle relative to the leg of the subject. A resting position may be a position in which a subject's leg is flatly extended on a supporting surface, such as a bed or pillow. For instance, a foot pedal of resistance device 224 may be positioned at about a 45 degree angle relative to a leg of a subject in a resting position. In other embodiments, foot pedals of resistance devices 224 may have adjustable angles. Controller 204 may be configured to adjust a force applied in direction R of one or both resistance devices 224. Controller 204 may increase or decrease a force applied by one of or both of resistance devices 128. In some embodiments, resistance devices 224 may include one or more sensors. Sensors of resistance devices 224 may include, but are not limited to, accelerometers, pressure sensors, inertial measurement units (IMU), and the like. Stimulation unit 208 may be configured to determine a location of a foot of a subject via a location of resistance device 224. In some embodiments, stimulation unit 208 may adjust a force applied via resistive devices 224 based on a determined location of a foot of a subject, such as increasing force, decreasing force, and the like.

Controller 204 may be in communication with one or more sensors of resistance devices 224 and may be configured to operate resistance devices 224 based on sensor data received. As a non-limiting example, a force applied by a subject on a resistance device 224 may be much greater than a force generated in direction R by the resistance device 224, to which controller 204 may increase a force of the resistance device 224 to compensate for the greater force generated by the subject. Controller 204 may provide a stimulation output that may combine pressures, electrical stimulation, and/or force through air bladders, stimulators 220, and/or resistance devices 224. In some embodiments, stimulation unit 208 may utilize an optimization algorithm to provide an optimized stimulation output based on data of one or more sensors. Optimization functions, may include, but are not limited to, loss functions, least squares optimization, Gradient descent optimization, Greedy algorithms, and/or other algorithms. In some embodiments, controller 204 may utilize an objective function. An “objective function” as used in this disclosure is a process of minimizing or maximizing one or more values based on a set of constraints. Controller 204 may generate an objective function to optimize a stimulation of a subject's body part. In some embodiments, an objective function performed by controller 204 may include an optimization criterion. An optimization criterion may include any description of a desired value or range of values for one or more attributes of stimulating a subject's body part. Desired values or ranges of values may include a maximal or minimal value, a range between maximal or minimal values, or an instruction to maximize or minimize an attribute and/or a threshold value. An optimization criterion may specify one or more desired stimulation criteria. As a non-limiting example, an optimization criterion may specific that resistance provided by resistance device 224 should be about 10 lbs while electrical stimulation applied to a patient should be about 120 mA. In an embodiment, an optimization criterion may assign weights to different attributes or values associated with attributes. “Weights”, as used herein, are be multipliers or other scalar numbers reflecting a relative importance of a particular attribute or value. One or more weights may be expressions of value to a subject of a particular outcome, attribute value, or other facet of stimulation. As a non-limiting example, minimization of an error margin may be multiplied by a first weight, while a tolerance of a stimulation parameter above a certain value may be multiplied by a second weight. Optimization criteria may be combined in weighted or unweighted combinations into a function reflecting an overall outcome desired by a person controlling the system or that is determined to be desired for the subject. Weights, desired values or ranges, and/or constraints may be set by a A person (e.g., medical professional, system controller, robot, or subject). In other embodiments, controller 104 may automatically set one or more weights, values or ranges, and/or constraints based on previous processing.

Referring now to FIG. 3, a front view of an attachable device 300 is illustrated. Attachable device 300 may have air bladders 304 and/or stimulators 308, which may be the same as that of air bladders 132 and stimulators 124 as described above with reference to FIG. 1. In some embodiments, stimulators 308 may be embedded in attachable device 300. In an embodiment, attachable device 300 may have eight stimulators 308, four in an upper portion of attachable device 300 and four in a lower portion of attachable device 300. Simulators 308 may be embedded into attachable device 300, which may help prevent subjects from attempting to remove stimulators 308. In some embodiments, two stimulators 308 may be positioned over quadricep muscles of a subject and two stimulators 308 may be positioned over a hamstring of a subject. In a lower portion of attachable device 300, in some embodiments, two stimulators 308 may be positioned over a tibialis anterior of a patient and another two stimulators 308 may be positioned over a gastrocnemius of the patient. In some embodiments, each stimulator 308 may be connected to one another via one or more connectors 332. Connectors 332 may be wires, leads, and/or other connectors, without limitation. Stimulators 308 may be grouped into sets, such as sets of two or more stimulators 308. In some embodiments, sets of stimulators 308 may have their own electrical channel which may be in communication with a controller or other device. As a non-limiting example, the two stimulators 308 positioned over the gastrocnemius of the patient described above may have a separate electrical channel than that of the two stimulators 308 positioned over the tibialis anterior of the patient.

In some embodiments, attachable device 300 may be attachable to a subject via attachment tabs 312. Attachment tabs 312 may include Velcro, hooks, loops, clips, and/or other attachment devices. In some embodiments, attachable device 300 may include four attachment tabs 312 or greater or less than four attachment tabs 312. Attachment tabs 312 may wrap around attachable device 300 and attach to a surface on an opposite side of attachment tabs 312, which may help secure attachable device 300 to a subject's limb. Attachment tabs 312 may be configured to attach to attachment surfaces 328. Attachment surfaces 328 may be Velcro, in some embodiments. In some embodiments, attachable device 300 may include knee hole 316. Knee hole 316 may be a cut-out of attachable device 300, in some embodiments. For instance, knee hole 316 may allow for a subject's knee to extend and retract while attachable device 300 is worn by a subject's leg. Attachable device 300 may have stimulators 308 above and/or below knee hole 316. In some embodiments, attachable device 300 may have air bladders 304 above and/or below knee hole 316.

Attachable device 300 may include air tubes 320. Air tubes 320 may be in fluidic communication with one or more air bladders 304 of attachable device 300. Air tube 320 may be configured to attach to an air outlet of a compression pump, such as described above with reference to FIGS. 1-2. In some embodiments, attachable device 300 may have three or more air tubes 320. In other embodiments, attachable device 300 may have less than three air tubes 320. Attachable device 300 may include leads 324. Leads 324 may be electrically conductive material that may provide an electrical connection to one or more stimulators 304. Leads 324 may be connectable to one or more external power supplies. In some embodiments, leads 324 may provide current, voltage, and the like to one or more stimulators 308. Leads 324 may be the same as stimulation cables 132 as described above with reference to FIG. 2, without limitation. Attachable device 300 may have stimulators 308 and air bladders 304 integrated into attachable device 300, which may allow for a “plug and play” operation of attachable device 300 with controllers, stimulations units, and the like.

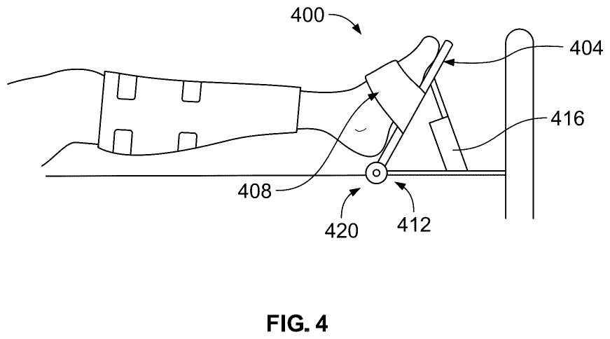

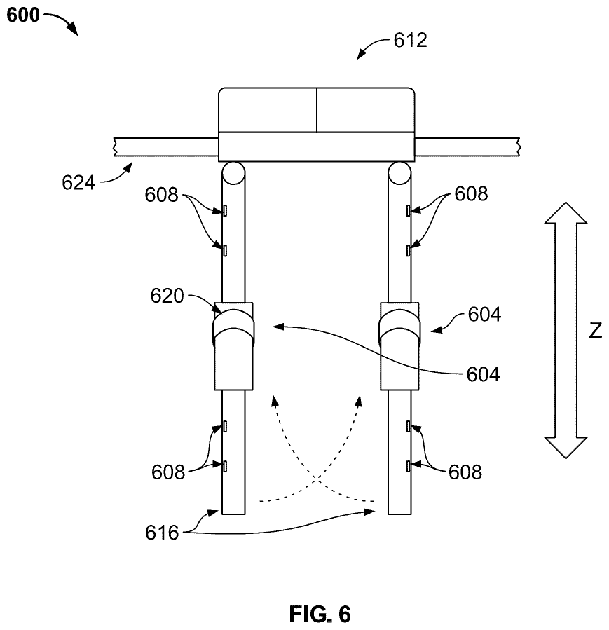

Referring now to FIG. 4, a side view of a resistance device in operation is shown. Resistance device 400 may be the same as resistance device 128 as described above with reference to FIG. 1. Resistance device 400 may include foot plate 404. Foot plate 404 may be made of a plastic, metal, or other material. Foot plate 404 may include strap 408. Strap 408 may secure a top portion of a subject's foot to foot plate 404. Strap 408 may be configured to secure a subject's foot to foot plate 404. Strap 408 and/or foot plate 404 may be adjustable in various positions. In some embodiments, foot plate 404 may be lockable in one or more positions. While locked, foot plate 404 may be configured to slide along a track of resistance device 400. While unlocked, foot plate 404 may provide resistance via a rotation of foot plate 404 about rotation point 420. Foot plate 404 may be operable to secure a foot of a subject in a prone position, in an embodiment. Foot plate 404 may be configured to slide along a track of resistance device 400, such as described below with reference to FIG. 6. In some embodiments, foot plate 404 may include rotation sensor 412. Rotation sensor 412 may include, but is not limited to, a rotary position sensor, gyroscope, and/or other sensor. Rotation sensor 412 may be configured to determine an angle of rotation of a subject's foot via rotation of foot plate 404, in some embodiments. Data generated by rotation sensor 412 may be used by a controller of resistance device 400 to determine a force generated by a subject's body part, such as a foot, ankle, or other body part. A controller of resistance device 400 may adjust resistance generated by resistance device 400 based on an amount of force determined to be generated by a subject's body part through rotation sensor 412.

Resistance device 400 may provide a force opposing a force generated by a subject's foot and/or leg. For instance, and without limitation, a one or more stimulators may activate a plantar or dorsal muscle group of a subject, to which resistance device 400 may oppose by providing a resistance through resistive element 416. Resistive element 416 may be a pneumatic brace, spring and/or other resistance device. In some embodiments, resistive element 416 may be a piston, such as a pneumatic, hydraulic, and/or spring piston. In some embodiments, resistive element 416 may be disposed between and coupled to footplate 404 and a portion of resistance device 400 that facilitates attachment to a footboard of a bed. Resistive element 416 may cause foot plate 404 to rotate about rotation point 420. Rotation point 420 may be a pivot or other component about which foot plate 404 may rotate. For instance, foot plate 404 may rotate in a counter-clockwise direction and/or clock-wise direction with respect to rotation point 320. Resistive element 416 may be configured to provide a force towards a subject's leg, in a counter-clockwise direction, and/or against a subject's leg, in a clockwise direction (with respect to the illustration shown in FIG. 4), through force applied to foot plate 404. As a non-limiting example, a subject may flex a dorsal muscle, which may bring the subject's foot towards their leg. Resistive element 416 may provide a force in a clockwise direction, opposite to the direction of movement of the subject's foot. This may provide a resistance to the dorsal muscles of the subject's foot. As another non-limiting example, a subject may activate their plantar muscles, which may move their foot away from their leg. Resistive element 416 may provide a force in a counter-clockwise direction, which may be opposite a movement of the subject's foot. A controller 104, such as described above with reference to FIG. 1, may control a movement of resistive element 416 based on sensor data, such as acceleration data, angular positioning data, and the like.

In some embodiments, a controller may be configured to determine a position of a heel of a subject. A controller may determine a position of a heel of a subject through data of sensor 412. Sensor data of sensor 412 may include, but is not limited to, angular data, rotational data, and/or other types of data. A controller may be configured to adjust force generated by resistance device 400 based on sensor data of sensor 412. In some embodiments, a controller may be configured to provide force through resistance device 400 to a subject in a closed-feedback loop with sensor data of sensor 412. In some embodiments, a closed-feedback loop may include a force generated by resistance device 400, an electrical stimulation via one or more stimulators, and/or pressure applied via one or more air bladders.

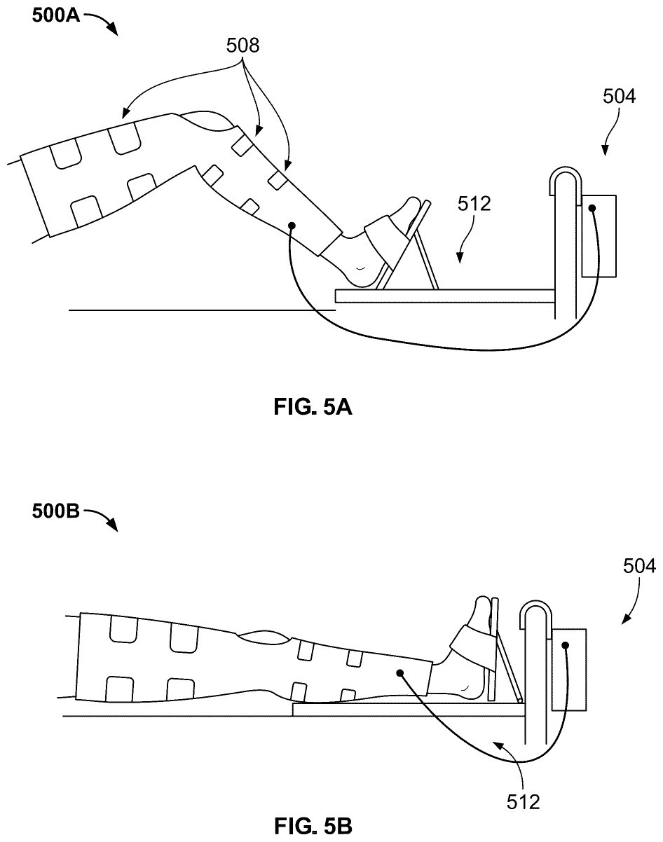

Referring now to FIG. 5A, an illustration of a system 500 for prevention of deep-vein thrombosis (DVT) edema or muscle atrophy in a subject's leg in a flexed position is shown. System 500 may include stimulation unit 504, stimulators 508 of an attachable device, and resistance device 512, each of which may be as described above with reference to FIGS. 1-2. Resistance device 512 may be a heel slide device and/or ankle flexion device, such as described above. Stimulation unit 504 may provide resistance against a subject's leg movement in conjunction with stimulation through one or more stimulators 508. In some embodiments, stimulation unit 504 may be configured to adjust air pressure of one or more bladders of an attachable device, activate one or more stimulators 508, and/or activate resistance device 512 which may provide a combination of stimulations on a subject's leg. In some embodiments, stimulators 508 may activate one or more muscles of a subject's leg which may cause the subject's leg to extend. Resistance device 512 may oppose an extension of a subject's leg caused by one or more stimulators 508. Stimulation unit 504 may control both an activation of one or more muscles of a subject's leg and a resistance provide by resistance device 512, in some embodiments. A sliding movement of resistance device 512 may be adjustable, for instance various distances of movement across a track of resistance device 512 may be adjustable. In some embodiments, one or more stoppers or other components may be positioned along a track of resistance device 512, which may restrict movement of a foot plate along the track.

Referring now to FIG. 5B, the system of FIG. 5A with a subject's leg extended is shown. Resistance device 512 may allow for a complete extension of a subject's leg. In some embodiments, stimulation unit 504 may stimulate a subject's legs via one or more stimulators 508 while the subject's leg is in an extended position. In some embodiments, stimulation unit 504 may activate resistance device 512 while a subject flexes their leg, which may provide a force opposing the subject's flexion of their leg. Stimulation unit 504 may cause one or more muscles of a subject's leg to contract, such as a hamstring of the subject, via one or more stimulators 508. Stimulation unit 504 may oppose a force generated by a contraction of a hamstring of a subject through resistance device 512.

Referring now to FIG. 6, a top view of a resistance device 600 is presented. Resistance device 600 may be a heel slide device and/or ankle flexion device. In some embodiments, resistance device 600 may be a stationary bike or a cycling portion on a bike ergometer. Resistance device 600 may be positioned adjacent to bed board 624. In some embodiments, resistance device 600 may attach to bed board 624 which may allow a positioning of resistance device 600 with respect to a patient in a bed. Resistance device 600 may be configured to apply force to two legs of a subject, in some embodiments. In some embodiments, resistance device 600 may include foot plates 604, sensors 608, and/or stimulation unit 612 which may be as described above with reference to FIGS. 1-4. In some embodiments, resistance device 600 may have a plurality of sensors 608 along tracks 616, such as along a first and second track in which a first foot plate 604 and a second foot plate 604 may be operable to slide along. Resistance device 600 may include foot straps 620, for instance a first foot strap 620 for a left foot and a second foot strap 620 for a right foot. Resistance device 600 may include rotation point, which may be as described above with reference to FIG. 4. Stimulation unit 612 may move foot plates 604 along tracks 616, such as in direction Z. In some embodiments, one or more stoppers may be embedded or positioned on tracks 616, which may restrict a movement of foot plates 604.

In some embodiments, sensors 608 may include linear sensors that may be configured to detect a position of a foot of a subject along tracks 616. A controller of stimulation unit 612 may be configured to provide resistance through force applied to foot plates 604 based on a closed-feedback loop of data received from one or more linear sensors of sensors 608.

Resistance device 600 may be attachable to bed board 628. Bed board 628 may be made of, but not limited to, wood, plastic, and/or other materials. Tracks 616 of resistance device 600 may be rotatable. For instance, tracks 616 may rotate 90 degrees from an initial position, such as 90 degrees away from bed board 628, towards legs of a subject. Tracks 616 may be placed under one or more legs of a subject. In some embodiments, tracks 616 may be rotatable about a hinge proximate to bed board 628, which may facilitate subject movement and/or installation of a subject leg in an attachable device and/or heel slide device. In some embodiments, tracks 616 may be used for both ankle flexion as described above with reference to FIG. 4 and leg extension as described above with reference to FIGS. 5A-B. In other embodiments, two sets of tracks 616 may be used, one set for leg extension and another set for ankle flexion.

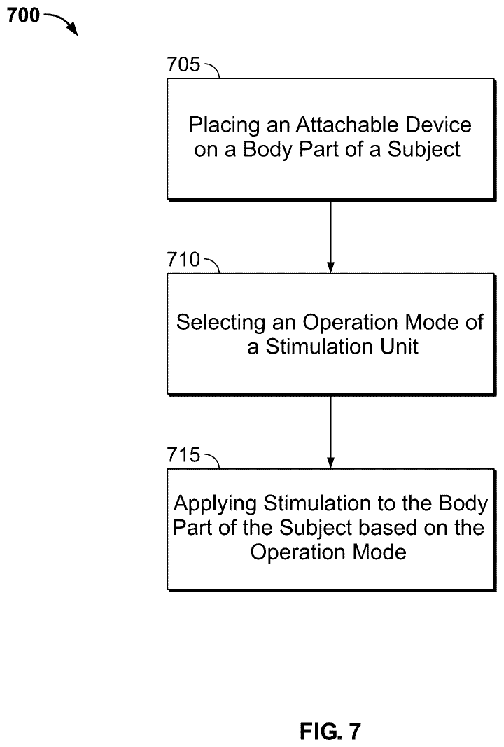

Referring now to FIG. 7, a flowchart 700 of a method of preventing deep-vein thrombosis (DVT) edema or muscle atrophy is presented. At step 705, method 700 includes placing an attachable device on a body part of a subject. An attachable device may include an electrode configured to stimulate a portion of the body part of the subject. In some embodiments, an attachable device may include a plurality of electrodes configured to stimulate various portions of the body part of the subject. In some embodiments, an attachable device may include an air bladder configured to apply pressure to a portion of the body part of the subject. An air bladder may be made of a flexible material, such as plastic or another material, without limitation. An attachable device may include two or more air bladders. In some embodiments, an attachable device may include an air inlet port fluidically connected to an air bladder. An air inlet port may be configured to connect to one or more external fluid sources. In some embodiments, an attachable device may have a first air bladder positioned at a top portion of the attachable device and a second air bladder positioned at a bottom portion of the attachable device. This step may be implemented, without limitation, as described above with reference to FIGS. 1-6.

At step 710, method 700 includes selecting an operation mode of a stimulation device of a controller in communication with the attachable device and in communication with a resistance device. A stimulation device may be in communication with one or more leads or other electrical connection components of an attachable device. In some embodiments, a controller may be configured to active an air bladder, stimulator, and/or resistance device. A mode of operation of a controller device may include, but is not limited to, periodic electrical stimulation, gradual air pressure increase or decreases, increases or decreases of force of one or more resistance devices, and/or other modes of operation. This step may be implemented, without limitation, as described above with reference to FIGS. 1-6.

At step 715, method 700 includes applying stimulation to the body part of the subject based on the operation mode through at least one electrode, applying pressure to the body part of the subject through an air bladder, or combination thereof. Applying stimulation may include activating one or more stimulators, air bladders, and the like of an attachable device. In some embodiments, applying stimulation to the body part of the subject may include applying a resistive force to the body part of the subject through one or more resistance devices. Resistance devices may include, but are not limited to, foot pedals, leg extension devices, leg retraction devices, and/or other devices. In some embodiments, applying stimulation includes applying a force to a body part of a subject through a resistance device. This step may be implemented, without limitation, as described above with reference to FIGS. 1-6.

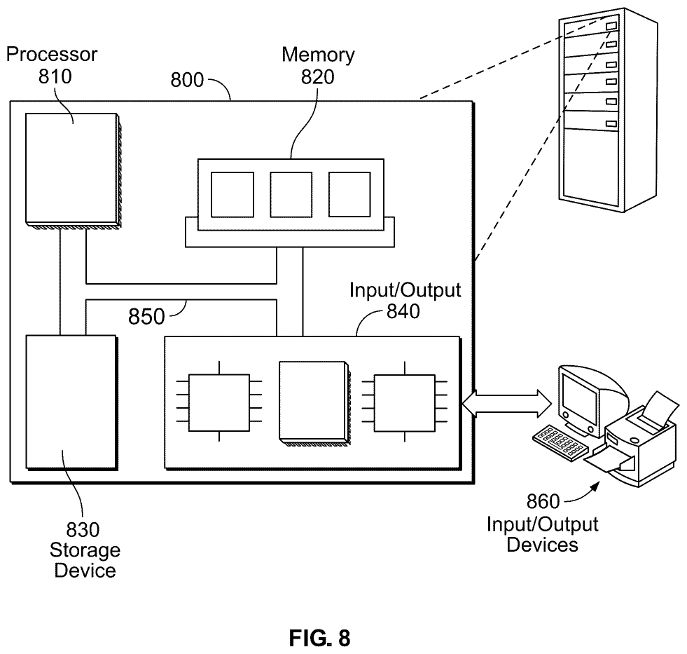

FIG. 8 is a block diagram of an example computer system 800 that may be used in implementing the technology described in this document. General-purpose computers, network appliances, mobile devices, or other electronic systems may also include at least portions of the system 800. The system 800 includes a processor 810, a memory 820, a storage device 830, and an input/output device 840. The apparatus may include disk storage and/or internal memory, each of which may be communicatively connected to each other. The apparatus 100 may include a processor 810. The processor 810 may enable both generic operating system (OS) functionality and/or application operations. In some embodiments, the processor 810 and the memory 820 may be communicatively connected. As used in this disclosure, “communicatively connected” means connected by way of a connection, attachment, or linkage between two or more elements which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. In some embodiments, the processor 810 may include any computing device as described in this disclosure, including without limitation a microcontroller, microprocessor, digital signal processor (DSP) and/or system on a chip (SoC) as described in this disclosure. The processor 810 may include, be included in, and/or communicate with a mobile device such as a mobile telephone or smartphone. The processor 810 may include a single computing device operating independently, or may include two or more computing device operating in concert, in parallel, sequentially or the like. Two or more computing devices may be included together in a single computing device or in two or more computing devices. The processor 810 may interface or communicate with one or more additional devices as described below in further detail via a network interface device. Network interface device may be utilized for connecting the processor 810 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. The processor 810 may include but is not limited to, for example, a computing device or cluster of computing devices in a first location and a second computing device or cluster of computing devices in a second location. The processor 810 may include one or more computing devices dedicated to data storage, security, distribution of traffic for load balancing, and the like. The processor 810 may distribute one or more computing tasks as described below across a plurality of computing devices of computing device, which may operate in parallel, in series, redundantly, or in any other manner used for distribution of tasks or memory between computing devices. The processor 810 may be implemented using a “shared nothing” architecture in which data is cached at the worker, in an embodiment, this may enable scalability of system 800 and/or processor 810.

With continued reference to FIG. 8, processor 810 and/or a computing device may be designed and/or configured by memory 820 to perform any method, method step, or sequence of method steps in any embodiment described in this disclosure, in any order and with any degree of repetition. For instance, the processor 810 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The processor 810 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Each of the components 810, 820, 830, and 840 may be interconnected, for example, using a system bus 850. The processor 810 is capable of processing instructions for execution within the system 800. In some implementations, the processor 810 is a single-threaded processor. In some implementations, the processor 810 is a multi-threaded processor. In some implementations, the processor 810 is a programmable (or reprogrammable) general purpose microprocessor or microcontroller. The processor 810 is capable of processing instructions stored in the memory 820 or on the storage device 830.

The memory 820 stores information within the system 800. In some implementations, the memory 820 is a non-transitory computer-readable medium. In some implementations, the memory 820 is a volatile memory unit. In some implementations, the memory 820 is a nonvolatile memory unit.

The storage device 830 is capable of providing mass storage for the system 800. In some implementations, the storage device 830 is a non-transitory computer-readable medium. In various different implementations, the storage device 830 may include, for example, a hard disk device, an optical disk device, a solid-date drive, a flash drive, or some other large capacity storage device. For example, the storage device may store long-term data (e.g., database data, file system data, etc.). The input/output device 840 provides input/output operations for the system 800. In some implementations, the input/output device 840 may include one or more network interface devices, e.g., an Ethernet card, a serial communication device, e.g., an RS-232 port, and/or a wireless interface device, e.g., an 802.11 card, a 3G wireless modem, or a 4G/5G wireless modem. In some implementations, the input/output device may include driver devices configured to receive input data and send output data to other input/output devices, e.g., keyboard, printer and display devices 860. In some examples, mobile computing devices, mobile communication devices, and other devices may be used.

In some embodiments, the systems provided for herein can be used in, or for, methods of preventing or ameliorating deep-vein thrombosis (DVT) edema or muscle atrophy of a subject. In some embodiments, the systems provided for herein can be used in, or for, methods of stimulating a muscle or movement of a body part of the subject.

As used herein, the term “ameliorating” refers to relieving the symptoms of a disease, disorder or condition in a subject already exhibiting the symptoms of the disease, disorder and/or condition. For example, ameliorating deep-vein thrombosis (DVT) edema in a subject that may already have such condition means that the subject's DVT edema does not worsen or that the condition improves. Improvement can be, for example, that the number of clots does not increase in the subject that the system is utilized with. In another example, if a subject is already showing symptoms of muscle atrophy, the atrophy does not worsen when the systems provided for herein are utilized with the subject. In some embodiments, the use of the systems provided for herein with a subject already showing symptoms of muscle atrophy can show increases in muscle mass. Therefore, in some embodiments, the systems provided for herein can be utilized in methods of either maintaining or increasing muscle mass in a subject or a subject's body part.

In some embodiments, the methods comprise placing an attachable device on a body part of the subject, the attachable device comprising at least one electrode configured to stimulate a portion of the body part of the subject. In some embodiments, the methods comprise selecting an operation mode of a stimulation unit of a controller in communication with the attachable device and in communication with a resistance device. In some embodiments, the methods comprise applying stimulation and resistance to the body part of the subject through the at least one electrode and resistance device based on the operation mode.

In some embodiments, applying stimulation comprises inflating or deflating the air bladder of the attachable device.

In some embodiments of the method provided for herein, selecting the operation mode comprises selecting an exercise mode. In some embodiments, upon selection of the exercise mode the stimulation unit activates the at least one electrode and shuts off the air bladder of the attachable device.

In some embodiments, the methods comprise detecting a movement of the body part of the subject through a sensor of a resistance device in communication with the controller; and activating, by the stimulation unit, the at least one electrode in a closed feedback loop based on the detected movement.

In some embodiments, the methods comprise providing variable resistance to the body part of the subject through the resistance device in communication with the controller.

In some embodiments, the methods comprise calculating a power output of a movement of the body part of the subject through a sensor of the resistance device in communication with the controller.

In some embodiments, the methods comprise adjusting a resistance level of the resistance device in communication with the controller.

In some embodiments of the methods provided for herein applying stimulation comprises or further comprises applying stimulation to a second leg of a subject through a second cuff in communication with the stimulation unit.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 830 may be implemented in a distributed way over a network, for example as a server farm or a set of widely distributed servers, or may be implemented in a single computing device.

Although an example processing system has been described in FIG. 8, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, a data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

A person (e.g., medical professional, system controller, robot, or subject) may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The terms “about” or “substantially” that modify a condition or relationship characteristic of a feature or features of an embodiment of the disclosure, are to be understood to mean that the condition or characteristic is defined to within tolerances that are acceptable for operation of the embodiment for an application for which it is intended. For example, the terms “about,” “substantially,” and/or “close” with respect to a magnitude or a numerical value may imply to be within an inclusive range of −10% to +10% of the respective magnitude or value.

It must be noted that, as used in the specification, the singular forms “a,” “an” and “the” include plural referents unless the context clearly dictates otherwise. By way of example, “an analogue” means one analogue or more than one analogue.

Each numerical value presented herein is contemplated to represent a minimum value or a maximum value in a range for a corresponding parameter. Accordingly, when added to the claims, the numerical value provides express support for claiming the range, which may lie above or below the numerical value, in accordance with the teachings herein. Every value between the minimum value and the maximum value within each numerical range presented herein (including in the figures), is contemplated and expressly supported herein, subject to the number of significant digits expressed in each particular range. Absent express inclusion in the claims, each numerical value presented herein is not to be considered limiting in any regard.

Having described certain embodiments of the disclosure, it will be apparent to those of ordinary skill in the art that other embodiments incorporating the concepts disclosed herein may be used without departing from the spirit and scope of the disclosure. Accordingly, the described embodiments are to be considered in all respects as only illustrative and not restrictive. The terms and expressions employed herein are used as terms and expressions of description and not of limitation and there is no intention, in the use of such terms and expressions, of excluding any equivalents of the features shown and described or portions thereof. The structural features and functions of the various embodiments may be arranged in various combinations and permutations, and all are considered to be within the scope of the disclosure. Unless otherwise necessitated, recited steps in the various methods may be performed in any order and certain steps may be performed substantially simultaneously and/or in parallel.

1. A system for prevention or amelioration of deep-vein thrombosis (DVT) edema or muscle atrophy, or for stimulating a muscle or stimulating the movement of a body part of a subject, the system comprising:
an attachable device, the attachable device comprising:
at least one electrode configured to stimulate a portion of the body part of the subject;
an air bladder configured to apply pressure to the portion of the body part of a subject; and
an air inlet port fluidically connected to the air bladder; and
a controller in electrical communication with the attachable device, the controller comprising:
a stimulation unit, wherein the stimulation unit is configured to stimulate and/or move at least a portion of the body part of the subject through activation of at least one of the at least one electrode;
a compression pump; and
at least one air tube connecting an outlet of the compression pump to the air inlet port of the attachable device, wherein the controller is configured to active the at least one electrode, compression pump, or both.

2. The system of claim 1, wherein the stimulation unit is further configured to activate the at least one electrode based on data generated from a sensor of the attachable device in a closed loop algorithm.

3. The system of claim 1, wherein the attachable device further comprises a resistance device, wherein the resistance device is configured to apply a force to the portion of the body part of the subject.

4. The system of claim 3, wherein the resistance device is one of a pneumatic, hydraulic, or spring piston.

5. The system of claim 3, wherein the resistance device includes a sensor configured to determine a position of a foot of the subject.

6. The system of claim 3, wherein the resistance device is a heel or ankle flexion device.

7. The system of claim 3, wherein the exercise unit includes a track with a footplate, the footplate operable to slide along the track.

8. The system of claim 7, wherein the track includes a linear sensor configured to determine a position of the footplate on the track.

9. The system of claim 8, wherein the stimulation unit is further configured to provide resistance through the resistance device based on data generated from the linear sensor in a closed feedback loop.

10. The system of claim 1, wherein the controller is configured to operate the stimulation unit in one of a plurality of operation modes.

11. A method of preventing or ameliorating deep-vein thrombosis (DVT) edema or muscle atrophy of a subject or a method of stimulating a muscle or movement of a body part of the subject, the method comprising:
placing an attachable device on a body part of the subject, the attachable device comprising:
at least one electrode configured to stimulate a portion of the body part of the subject;
an air bladder configured to apply pressure to the portion of the body part of the subject; and
an air inlet port fluidically connected to the air bladder;
selecting an operation mode of a controller in communication with the attachable device; and
applying stimulation to the body part of the subject through the at least one electrode, applying pressure to the body part through the air bladder, or both, based on the operation mode.

12. The method of claim 11, wherein applying stimulation further comprises inflating or deflating the air bladder of the attachable device.

13. The method of claim 11, further comprising applying force to the body part of the user through a resistance device in communication with the controller based on the operation mode.

14. The method of claim 13, wherein the resistance device includes one of an ankle flexion device or a heel slide device.

15. The method of claim 13, wherein selecting the operation mode comprises selecting an exercise mode, wherein upon selection of the exercise mode the stimulation unit activates the at least one electrode and applies a force to the body part of the subject through the resistance device.

16. The method of claim 13, further comprising:
detecting a movement of the body part of the subject through a sensor of the resistance device in communication with the controller; and
activating, by the stimulation unit, the at least one electrode in a closed feedback loop based on the detected movement.

17. The method of claim 13, further comprising providing variable resistance to the body part of the subject through the resistance device.

18. The method of claim 13, further comprising calculating a force of a movement of the body part of the subject through a sensor of the resistance device in communication with the controller.

19. The method of claim 18, further comprising adjusting a resistance level of the resistance device based on the force.

20. The method of claim 11, wherein the portion of the body part of the subject is a leg.
